(19) United States
(12) Patent Application Publication
(10) Pub. No.: US 2009/0317451 A1
(43) Pub. Date: Dec. 24, 2009

(54) PRESSURE-SENSITIVE ADHESIVE FOR SKIN SURFACE AND/OR TRANSDERMAL SUBSTANCE DELIVERY

(76) Inventor: Ray L. Hauser, Boulder, CO (US)

Correspondence Address: COCHRAN FREUND & YOUNG LLC, 2026 CARIBOU DR, SUITE 201, FORT COLLINS, CO, 80525, US

(21) Appl. No.: 12/548,301

(22) Filed: Aug. 26, 2009

Related U.S. Application Data
(60) Provisional application No. 61/091,912, filed on Aug. 26, 2008.

(51) Int. Cl. A61F 13/02; A61K 47/30; A61K 31/60

(52) U.S. Cl. 424/448; 514/772.3; 514/165

(57) ABSTRACT

A pressure-sensitive adhesive composition is described having a reservoir of medication or other substance and capable of intimate contact with a target area of skin, rapid conveyance of the medication onto and/or into the target area, and ready removal from the area of skin using water. Polyvinylpyrrolidone and glycerol mixtures have been found to dissolve a large number of medications while producing a self-tackifying, pressure-sensitive adhesive composition.

### RELATED CASES

The present application claims the benefit of provisional patent application Ser. No. 61/091,912 for “Pressure-Sensitive Adhesive For Surface Or Transdermal Substance Delivery” by Ray L. Hauser, filed on 26 Aug. 2008, which provisional application is hereby incorporated by reference herein for all that it discloses and teaches.

### FIELD OF THE INVENTION

The present invention relates generally to the introduction of medication into a patient and, more particularly, to a pressure-sensitive adhesive composition for skin surface and/or transdermal delivery of medication or other substances to a patient.

### BACKGROUND OF THE INVENTION

Pressure-sensitive adhesives generally include a combination of an elastomer, a plasticizer and a tackifying resin. Natural rubber, synthetic hydrocarbon elastomers, silicone elastomers and acrylic elastomers are the most common rubbery components. Oils or plasticizers are used to swell the elastomers and to make them more soft and stretchy, giving “legs” to the adhesive mix. Resins are generally hard thermoplastics that are soluble in the plasticizer and provide shear strength and limit the stretchiness of the final adhesive. Solvents are often used to decrease viscosity of the mix so that a thin layer of the adhesive can be applied to a substrate or carrier. If the original elastomer is in a latex or emulsion form, the plasticizer and/or resin may also be emulsified. Useful elastomers and plasticizers have low Hildebrand solubility parameters, usually less than 9.5 [cal/cc]^{1/2} (2.045 [cal/cc]^{1/2}=MPa^{0.5}).

In order for a medication to be most effective, it must be placed in intimate contact with the target area. Conventionally, a salve or cream medication must first be placed on the problem area or injury, followed by a bandage. Band-Aid® type bandages have occasionally been medicated, but the medication fails to attach to the site of the injury and does not provide direct medication thereto. Similarly, medical bandages usually provide a covering for an injured area, but are generally loose coverings and permit ingress of dirt and germs to an area that should be kept clean.

Medicated patches are used to provide dosages to the body, where the medication is contained within a pressure-sensitive adhesive. Nicotine patches are a common example of this type of application, and formulation of such patches requires that the medication dissolve in and otherwise be compatible with the adhesive and its plasticizer, which are usually relatively non-polar and have low solubility parameters. The use of polar solvents as carriers for disinfectants, painkillers fungicides, etc. broadens the spectrum of medications that can be used both topically and systematically. Plasticizers having low solubility parameters generally also yield poor permeation rates into and through human skin.

Additives for enhancing permeation rates through the skin have been described. These often use cell envelope disruptive compounds such as isopropyl myristate, methyl laurate, oleic alcohol, fatty glycerol esters and fatty sorbitan esters. These compounds may be incorporated in mono-, di- or tri-ols.

The use of transdermal compositions for controlling medicament delivery through the skin at a substantially constant rate is known. Such delivery systems may incorporate a medicament into a carrier such as a polymeric matrix and/or a pressure-sensitive adhesive formulation, as examples, whereby the pressure-sensitive adhesive effectively adheres to the skin, thereby permitting infusion of the medicament from the carrier through the skin and into the bloodstream of a patient. Blends of rubber-based pressure-sensitive adhesives, such as polysiloxane, with polymers having different solubility parameters, including a soluble polyvinylpyrrolidone as an example, have been used to adjust the solubility of the drugs in such blends, thereby controlling the delivery of the drug from the system through the dermis. A fabric backing material or plastic film having a thin layer of adhesive on the dermal side are commonly used for such delivery systems.

Polymeric diffusion matrices comprising a polar plasticizer, polyvinyl alcohol, and polyvinylpyrrolidone have been described as being effective for the sustained transdermal release of pharmaceutically useful amounts of propranolol, phenylephrine, clonidine, terbutaline, and phenylpropanolamine contained therein, but have been assessed as not having significant pressure-sensitive adhesive properties and as not providing identifiable adhesion to the skin.

Sustained-release film dressings having attachment properties to human skin for healing wounds by releasing epidermal growth factor have also been described. Such compositions include the polymer chitosan; one or more viscosity modifiers such as hydroxypropylmethylcellulose, gellan gum, pullulan, etc. as a film base for consistently releasing the main ingredient; an antioxidant such as EDTA, vitamin C, etc. as a stabilizing agent; and glycerin, propylene glycol, polyvinyl alcohol, polyvinylpyrrolidone, and polyethylene glycol, etc. as plasticizers.

A pressure-sensitive adhesive gel including polyvinylpyrrolidone, greater than 2% by weight of polyvinyl alcohol, a humectant, water, and an ionic species or a drug has also been described.

### SUMMARY OF THE INVENTION

Accordingly, it is an object of the present invention to provide a pressure-sensitive adhesive composition having a reservoir of medication.

Another object of the invention is to provide a pressure-sensitive adhesive composition capable of intimate contact with a target area of external skin or internal tissue.

Yet another object of the invention is to provide a pressure-sensitive adhesive composition capable of rapid conveyance of a medication to a target area of skin.

Still another object of the invention is to provide a pressure-sensitive adhesive composition capable of being painlessly removed using water.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

To achieve the foregoing and other objects, and in accordance with the purposes of the present invention, as embodied and broadly described herein, the pressure-sensitive adhesive composition hereof, consists essentially of: polyvinylpyrrolidone, at least one polar plasticizer and at least one substance to be delivered onto the skin or transdermally thereto.

In another aspect of the invention, and in accordance with its objects and purposes, the pressure-sensitive adhesive composition hereof, includes: polyvinylpyrrolidone as the principal pressure-sensitive adhesive, at least one polar plasticizer and at least one substance to be delivered onto the skin or transdermally thereto.

In yet another aspect of the invention, and in accordance with its objects and purposes, the method for delivering a substance onto the surface of skin or transdermally thereto, hereof, includes the steps of: forming a mixture of the at least one substance with a pressure-sensitive adhesive composition consisting essentially of: polyvinylpyrrolidone, and at least one polar plasticizer; and applying the mixture to the surface of the skin.

In still another aspect of the invention, and in accordance with its objects and purposes, the method for delivering a substance onto the surface of skin or transdermally thereto, hereof, includes the steps of: forming a mixture of the at least one substance with a pressure-sensitive adhesive composition including: polyvinylpyrrolidone as the principal pressure-sensitive adhesive, and at least one polar plasticizer; and applying the mixture to the surface of the skin.

In another aspect of the invention, and in accordance with its objects and purposes, the pressure-sensitive adhesive composition, hereof, includes: polyvinylpyrrolidone, less than 2% by weight of polyvinyl alcohol, at least one polar plasticizer and at least one substance to be delivered onto skin or transdermally thereto.

In yet another aspect of the invention, and in accordance with its objects and purposes, the method for delivering a substance onto the surface of skin or transdermally thereto, hereof, includes the steps of: forming a mixture of the at least one substance with a pressure-sensitive adhesive composition including: polyvinylpyrrolidone, less than 2% by weight of polyvinyl alcohol, and at least one polar plasticizer; and applying the mixture to the surface of the skin.

In still another aspect of the invention, and in accordance with its objects and purposes, the pressure-sensitive adhesive composition, hereof, includes between 25 wt. % and 70 wt. % of polyvinylpyrrolidone; between 10 wt. % and 50 wt. % of at least one plasticizer having a solubility parameter exceeding 20 MPa^{0.5} and a normal boiling temperature exceeding 150° C.; between 2 wt. % and 15 wt. % of water; and greater than 1% of at least one chelating agent.

Benefits and advantages of the present invention include, but are not limited to, providing a pressure-sensitive adhesive having a reservoir of medication and capable of intimate contact with a target area of skin, rapid conveyance of a medication to the target area, and ready removal from the area of skin using water.

### DETAILED DESCRIPTION OF THE INVENTION

Briefly, the present invention includes pressure-sensitive adhesive compositions of matter including polyvinylpyrrolidone (PVP), a polar plasticizer or a mixture of polar plasticizers, such as glycerol, propylene glycol, and propylene carbonate, and mixtures thereof, and an effective amount of medication or other substance, for providing a medicated layer having a reservoir of medication or other substance, with intimate contact to the target area of the body, and with rapid conveyance of a medication to the target. Less than 2% by weight of polyvinyl alcohol may be used in some of the compositions of matter hereof, and PVP is utilized as the principal pressure-sensitive adhesive.

Polyvinylpyrrolidone (Chemical Abstracts Numbers 9003-39-8, 9080-59-5, and 84057-81-8) is a thermoplastic that is self-tackifying when dissolved in some solvents. The term “polyvinylpyrrolidone” or PVP, as used herein, refers to a polymer, either a homopolymer or copolymer, containing N-vinylpyrrolidone as the monomeric unit. Typical PVP polymers are homopolymeric PVPs known in the pharmaceutical industry under a variety of designations including Povidone, Polyvidone, Polyvidonum soluble, and Poly(1-vinyl-2-pyrrolidone). The term “soluble” when used with reference to PVP means that the polymer is soluble in water and/or alcohols and is generally not substantially cross-linked.

As used in this invention, PVP may also include copolymers of polyvinylpyrrolidone and mixtures of this polymer with other water and/or alcohol-soluble polymers such as poly(ethyl oxazoline), polyhydroxy ether, poly(ethylene oxide), poly(ethenyl formamide), and polyvinyl alcohol, as examples.

A relatively nonvolatile solvent can dissolve the PVP as a plasticizer to give a pressure-sensitive adhesive whose physical properties are dependent upon the ratio of solid and liquid. PVP is available in molecular weights ranging between about 1×10⁴ and about 1.3×10⁶ Daltons, the higher molecular weights yielding stronger, stiffer pressure-sensitive adhesives.

PVP has a solubility parameter about 11.0 [cal/cc]^{1/2} (22.5 MPa^{0.5}) and it is readily soluble in polar solvents such as water, alcohols, polyols, and alkyl carbonates. Since most uses for a pressure-sensitive adhesive are for applications where water solubility would be a disadvantage, this polymer has been largely overlooked by the trade. Masking tapes, duct tape, and medical bandages normally require a moderate degree of resistance to water. The water resistance of PVP can be increased by cross-linking this polymer by radiation or chemical means before or after dissolution in the plasticizer/solvent.

Tapes and bandages that can be removed readily and painlessly by wetting an adhesive, are often required, and PVP is suitable for such applications. Bandages covering thermal or chemical burns, and bandages on very sensitive skin typify this requirement. Infants and elderly persons have sensitive skin that is pained or injured by removal of conventional pressure-sensitive adhesive bandages.

One suitable plasticizer, glycerol (glycerin), has a solubility parameter of about 16.5 [cal/cc]^{1/2} (33.7 MPa^{0.5}). The PVP/glycerol formulation of the present invention inherently has high rate of permeation by virtue of the glycerol content, and it generally needs no further enhancement.

PVP and glycerol are GRAS (Generally Recognized as Safe) chemicals. PVP has been used as a component of blood plasma. Glycerol has been used as an internal medication and as a component of many cosmetics for many years. The present invention uses glycerol both as a plasticizer for the PVP and as a solvent and mobile medium for transferring the medication through the skin.

The word “substance” as used herein includes medications, adjuvants and biologicals, such as growth factors, proteins, enzymes, antagonists, immune-modifying materials, and chelates, as examples.

Adhesive patches based upon PVP can be made using relatively little organic solvent, thus minimizing the amount of volatile organic compounds emitted during application of the adhesive to a release paper or to a backing, or with water solvent, also, for easy spreading onto a backing material. Alternatively, the adhesive can be applied as a plasticized melt at about 100° C. with zero or minimal emissions to the environment. Backing materials may include biodegradable microporous polymeric materials.

Where increased shear strength is required for the PVP adhesive, cross-linking the polymer with persulfate or with boric acid is effective. It is believed by the present inventor that addition of vinyl ester resins, polyethylene oxide, polyethylene glycol, polypropylene glycol, polyhydroxyether, poly(ethyl)oxazoline, and natural rosin will also increase the shear strength of the plasticized PVP solutions.

Medical terms used in the specification and claims of this invention are defined in TABLE 1.

TABLE 1
DEFINITION OF TERMS

| Category of | | |
|---|---|---|
| Additive | Definition of Additive | Examples of Additives* |
| Medication | A medicinal substance, medicament | All of the following categories |
| Analgesic | A drug used to relieve pain | Acetaminophen |
| | | Aspirin |
| | | Benzocaine |
| | | Fentanyl |
| | | Ibuprofen |
| | | Ketoprofen |
| | | Lidocaine |
| | | Reaction product of triethanolamine with |
| | | acetylsalicylic acid |
| | | Trolamine |
| Antiseptic | Any liquid or chemical substance | Bacitracin |
| | which is used to inhibit the growth of | Bactrim |
| | germs or to actually destroy germs, | Doxycycline hyclate |
| | whether bacteria or virus | Erythromycin hydrate |
| | | Iodine |
| | | Metronidazole |
| | | Neomycin |
| | | Polymyxin β hydrochloride |
| | | Trimethoprimsulfamethoxazole |
| | | Zinc oxide and zinc salts |
| | | ZnO-Acetyl salicylic acid |
| | | reaction product |
| Antiviral | A chemical substance which inhibits | Acyclovir |
| | growth or destroys virus | Bleomycin glycol-peptide |
| | | Famciclovir |
| | | Imiquimod |
| | | Interferon α protein |
| | | Penciclovir |
| Cationic antiseptic | Salt of metallic element | Calcium alginate |
| | | Copper salt |
| | | Silver salt |
| | | Silver sulfadiazine |
| | | Zinc salt |
| Chelation Therapy | Chelate | Salt of ethylenedinitrilotetraacetic acid |
| Chemotherapy | Chemical use to treat disease or | Albuterol |
| | mental illness | Alendronate sodium |
| | | Alprazolam |
| | | Cisplatin |
| | | Curcumin |
| | | Doxorubicin |
| | | Etanercept |
| | | Fenoldopam |
| | | 5-fluorourasil |
| | | Haloperidol |
| | | Hyaluronic acid |
| | | Paclitaxel |
| | | Papavrine |
| | | Pilocarpine |
| | | Promethazine |
| | | Scopalamine |
| | | Taxanes |
| | | Verapamil |
| Cortisone | A family of glucocorticoid | Cortisone |
| | compounds and derivatives | Hydrocortisone |
| | | Prednisone |
| Fungicide | An agent that inhibits or destroys | Cupric gluconate |
| | fungi | Cupric tartrate |
| | | Cyclopirox |
| | | Ketoconazole |
| | | Urea |
| Hormone | Natural or synthetic glandular | Albuterol |
| | chemical | Estradiol |
| | | Estrogen |
| | | Norethindrone acetate |
| | | Testosterone |
| Oxidizer, NO | A chemical that reacts to cause an | Benzoyl peroxide |
| provider | increase of valence | Calcium nitrate |
| | | Iodine |
| | | Nitroglycerine |
| | | Silver nitrate |
| | | Sodium nitrate |
| Treatments for | Soothing chemicals, acid | Aloe Vera |
| topical poisons and | neutralizers or habit-reduction | Camphor |
| skin ailments | treatments | Honey |
| | | Imiquimod |
| | | Nicotine |
| | | Reaction product of triethanolamine |
| | | with acetyl salicylic acid |
| | | Reaction product of zinc oxide with |
| | | acetyl salicylic acid |
| | | Trolamine |

*Examples include salts, acids, esters and derivatives thereof as listed in The Merck Index, which meet the solubility criteria of this invention. Preservatives may be of benefit in some medicated formulas of the present invention.

Reference will now be made in detail to the present embodiments of the invention. A medicated patch using PVP and glycerol is inherently permeable to mass transfer of water away from the body and it is moderately permeable to transport of air if the backing is likewise permeable. These are desirable characteristics for medicated patches, and PVP is better in this regard than are conventional pressure-sensitive elastomers. This adhesive is also less irritating to the skin when in contact therewith for multiple days than most conventional rubber-based adhesives.

The dissolution of PVP in glycerols is accomplished by gradual addition of the PVP powder to the plasticizer or plasticizer/alcohol solution at room temperature with stirring. Warming can facilitate complete dissolution of undissolved PVP which is evidenced by clarity of solution. Medications or other substances may be added to the glycerol/alcohol solution before or after making the PVP solution. Methanol, denatured ethanol, isopropanol, and water, and combinations thereof may be appropriate solvents for preparing a solution of moderate viscosity effective for application to a release paper or to a backing material.

Having generally described the invention, the following EXAMPLES provide additional details:

### Example 1

TABLE 2 is a list of formulations that were prepared in which the medication was observed to be soluble within the plasticizer and which gave good pressure-sensitive adhesive characteristics. Patches were made with some of these, often using alcohol as a volatile diluent to facilitate spreading of the adhesive on a substrate. In TABLE 2, all quantities are in grams, PVP is polyvinyl pyrrolidone, Prop Glycol is propylene glycol, AcSA is acetyl salicylic acid, and TEOA is triethanolamine. The solubility parameters of the plasticizers are Glycerol 33.7, Propylene Glycol 30.6, Propylene Carbonate 27.1, and Dipropylene Glycol 31.7 MPa^{0.5}.

TABLE 2
FORMULATIONS HAVING CONFIRMED GOOD ADHESION
AND GOOD SOLUBILITY OF MEDICATION

| PVP Molecular | | | | |
|---|---|---|---|---|
| Weight | | | | |
| 40,000 | 1,300,000 | Plasticizer | Medication | Treatment |
| 10 | | Prop.Glycol 10 | Aloe Vera 40:1 10 | For itch prevention, |
| | | | | effective 2-3 weeks |
| | | | | Tacky, low viscosity |
| 12 | | Glycerol 10 | Bacitracin 0.2 | Antibiotic 0.7% |
| | | Prop Glycol 2 | | |
| 12 | | Glycerol 10 | Acetominadophenol | Analgesic |
| | | Prop Glycol 2.7 | 0.5 | |
| 12 | | Glycerol 10 | Prednisone 0.1 | Anti-inflammatory |
| | | Prop Glycol 20 | | |
| 10 | | Glycerol 20 | Calcium nitrate-4, | Anti-itch, actinic |
| | | | hydrate 2 | keratoses |
| 10 | | Glycerol 10.7 | Aspirin pill dissolved | Analgesic |
| | | Prop Glycol 5.2 | in isopropanol, filtered | |
| | | Prop Carbonate | | |
| | | 1.1 | | |
| 10 | | Glycerol 10 | Ibuprofen 0.2 g | Analgesic |
| | | Prop Carbonate 3 | Methyl salicylate 1.2 | |
| 5 | | Glycerol 20 | Aspirin pill dissolved | Analgesic |
| | | Prop Glycol 8 | in isopropanol, filtered | |
| 10 | 2 | Prop Carbonate | Aspirin pill dissolved | Analgesic |
| | | 10 | in isopropanol, filtered, | |
| | | | 0.52 gm aspirin | |
| 10 | 2 | Glycerol 20 | Calcium nitrate-4 | Anti-itch |
| | | | hydrate 1 | |
| 5 | | Glycerol 10 | Hydrocortisone 0.1 | Anti-inflammatory |
| | | Diprop Glycol | | |
| | | 4.9 | | |
| 6 | | Glycerol 20.8 | Camphor 1 | Anti-itch |
| 20 | | Glycerol 25 | Ketoprofen 0.5 | Analgesic |
| | | Prop Glycol 10 | | |
| 10 | 5 | Glycerol 25 | Reaction Product of | Analgesic |
| | | | AcSA and TEOA 55 | |
| | 2.5 | Glycerol 12.5 | Reaction Product of | Analgesic |
| | | | AcSA and TEOA 20 | |
| | 5 | Glycerol 20 | Ketoprofen 0.5 | Analgesic |
| | 10 | Glycerol 20 | Benzocaine 1 | Antibiotic |
| | 5 | Glycerol 10 | Silver Nitrate 0.5 | Antibiotic |
| | 5 | Glycerol 10 | AcSA 7.1 | Analgesic, Acid |
| | | | TEOA 2.9 | neutralizer |
| | 5 | Glycerol 20 | Ketoprofen 0.5 | Analgesic |
| | | | Triton X100 10 drops | |
| | 12.5 | Glycerol 20 | AcSA 7.1 | Analgesic |
| | | | TEOA 2.9 | |
| | 10 | Glycerol 25 | Benzocaine 1 | Pain killer |
| | | | Triton X100 0.25 | |
| | 1.2 | Glycerol 2.5 | Glucosamine- | Local Arthritis |
| | | | Chondroiton 0.25 | Treatment |
| | 10 | Glycerol 20 | Iodine 2 | Bactericide |
| | 10 | Glycerol | Urea 5 | Subungual keratosis |
| | 15 | Glycerol 15 | CaNa₂EDTA 5 | Good adhesive |
| | | | Denatured Alcohol 30 | |
| | | | Cab-O-Sil M-5, 1 | |

Trolamine acetylsalicylate made from an equimolar mixture of triethanolamine with acetylsalicylic acid (aspirin) heated to about 175° F. for about one hour is a liquid that dissolves PVP, thereby permitting a medicated formulation with much higher concentration of this painkiller than is obtainable by dissolving aspirin in glycerol.

TABLE 2 lists medications, most of which are soluble in glycerol and/or water, for a variety of conditions, and notes formulations that have been prepared using the identified medications in solutions containing polar plasticizers and PVP. Propylene glycol, propylene carbonate, or glycerol triacetate are sometimes added to facilitate dissolution of the medication. These three solvent/plasticizers are suitable for use in direct contact with the skin, in limited quantities.

It is to be noted that the glycerol hinders the decomposition of aspirin by moisture, thereby giving longer shelf life to the product than would be otherwise obtained (Merck 12^{th} edition, #886). It is also noted that ketoprofen is reported to have a high rate of transpiration through human skin (“In Vitro Topical Delivery of Non-Steroidal Anti-inflammatory Drugs Through Human Skin,” Vincent, Laugel, Marty, Arzneimittelforschung, 1999 June; 49(6):509-13).

Many of the pressure-sensitive adhesives of TABLE 2 have been applied to a variety of backings including the cloth pad of Nextel™ bandages, and of Johnson & Johnson Band-Aids®, fabrics, nonwoven fabrics and microporous membranes. It is to be noted that many commercial bandages contain adhesives with little resistance to water, and that some stick sufficiently well that they pull hair and skin when removed. Some pressure-sensitive adhesives used in name-brand bandages leave a light pink, itchy skin after a week of contact. By contrast, there is no skin irritation generated by PVP adhesive compositions, and the PVP-based adhesives of the present invention can be removed painlessly and without damage to underlying tissue, by wetting with water. 3M Scotchgard™ water repellant has been applied to the backing of some bandages to increase resistance to external water permeation during washing or showering and have survived six showers over a 2-week period.

The adhesive of the present invention has also been applied to a thin (about 25 μm) and flexible porous film of polyvinylidenefluoride (PVDF). An almost transparent patch was obtained which may have merit for covering acne blemishes where an antibiotic can be combined with a painkiller or itch suppressant. A transparent or translucent medicated patch may permit observation of a wound through the patch to discern its healing progress

### Example 2

TABLE 3 is a list of medications that are expected to be effective substances for incorporation in the pressure-sensitive adhesive compositions of the present invention, based upon reported solubility in water, alcohol or similar polar solvents.

TABLE 3
MEDICATIONS EXPECTED TO BE EFFECTIVE FOR INCORPORATION
INTO AND SUCCESSFULLY DELIVERED BY MIXTURES OF
POLYVINYLPYRROLIDONE AND POLAR PLASTICIZERS

| | | Merck Reference, | |
|---|---|---|---|
| | | 12^{th} edition or other | Reported |
| Treatment | Medication | reference | Solubility |
| Acne | Benzoyl peroxide | 1149 | Alcohol, slight |
| Actinic keratosis | Silver nitrate | 8661 | Alcohol |
| Arterial bleeding | Chitosan, chitin sulfate, | 2105 | Alcohol |
| | chitosan lactic acid | | |
| Angina | Nitroglycerin | 6704 | Alcohol |
| | Isosorbide dinitrate | 5245 | Alcohol |
| Antibiotic | Netronidazole | 6242 | Water, alcohol |
| | Neomycin | 6542 | Water |
| | Polymyxin β- | 7734 | Alcohol |
| | hydrochloride | | |
| | Erythromycin hydrate | 3720 | Alcohol |
| | Bacitracin A | 965 | Alcohol |
| | Doxycylind hyclate | 3496 | Alcohol |
| Antibiotic for | Trimethoprim[sulfa- | 9086 | Alcohol |
| MRSA | methoxazol | | |
| Antiseptic | Silver nitrate | 8661 | Alcohol |
| | Iodine | 5034 | Alcohol |
| | Reaction product of zinc | 10279, 886 | Alcohol |
| | oxide with AcSA | | |
| Antiviral | Acyclovir | 148 | Alcohol |
| | Famciclovir | 3971 | Alcohol |
| | Penciclovir | 7216 | Water |
| | Interferon alpha protein | 5016 Martindale Drug | Water, alcohol |
| | | Reference | |
| | Bleomycin glycopeptice | 1351 | Water, methanol |
| | Imiquimod | 4957 | Glycerol + |
| | | | isostearic acid |
| Arthritis | Glyucosamine- | 4466 | Alcohol |
| | Chondroitin | | |
| | MSM, dimethyl sulfone | 3307 | Alcohol |
| | Etanercept | www.medicinenet.com | Water, alcohol |
| Asthma | Albuterol | 217 | Alcohol |
| Basal cells | Liquimod | 4957 | Alcohol |
| Burn injury | Silver sulfadiazine | 9071 | Alcohol, slight |
| | Calcium and zinc | 241 | Alcohol, slight |
| | alginates | | |
| Cancer, ovarian and | Paclitaxel | 7117 | Alcohol |
| lung | | | |
| Cancer | Doxorubicin | 3495 | Alcohol |
| | Cisplatin | 2378 | Dimethyl |
| | | | formamide |
| | Curcumin | 2744 | Alcohol |
| Cerebral Blood | Papavrine | 7151 | Alcohol |
| circulation | | | |
| Eczema | Aloe gel 40:1 | 312 | Alcohol |
| Fungus | Terbinafinehydrochloride | 9299 | Alcohol |
| | Ciclopirox | 2325 Martindale Drug | Alcohol |
| | | Reference | |
| | Ketoconazole | 5313 | Alcohol |
| | Cupric gluconate and | 2706 | Alcohol |
| | tartrate | | |
| Glaucoma | Pilocarpine | 7578 | Alcohol |
| Hormone therapy | Estrogen | 3751 | Alcohol slight |
| | Estradiol | 3476 | Alcohol |
| | Testosterone | 9322 | Alcohol |
| | Norethindrone acetate | 6790 | Alcohol |
| | Albuterol | 217 | Alcohol |
| Hypertension | Fenoldopam | 4020 | Alcohol |
| | Verapamil | 10083 | Alcohol |
| Itch | Camphor | 1779 | Alcohol |
| Nausea | Scopolamine | 8550 | Alcohol |
| Pain | Benzocaine | 1116 | Alcohol |
| | Lidocaine | 5505 | Alcohol |
| | Aspirin | 886 | Alcohol |
| | Ibuprofen | 4925 | Alcohol |
| | Ketoprofen | 5316 | Alcohol |
| | Acetaminophen | 45 | Alcohol |
| | Fentanyl | 4043 | Alcohol |
| | Trolamine | 9798 | Miscible liquid |
| | Reaction product of | Novel | Miscible liquid |
| | AcSA and TEOA | | |
| Poison ivy, oak, | Reaction product of | Novel | Miscible liquid, |
| sumac | AcSA and TEOA | | alkaline |
| Psychosis | Alprazolam | 320 | Alcohol |
| | Haloperidol | 4629 | Alcohol |
| Osteoarthritis | Hyaluronic acid, salts | 4793 | Alcohol |
| | and esters | | |
| Osteoporosis | Alendronic acid salts | 228 | Alcohol |
| Rheumatoid | Etanercept | | Glycerol |
| Arthritis | | | |
| Tattoo removal | Hydroxyquinol, 1,2,4- | 1101 | Glycerol |
| | benzotriol | | |
| Tobacco habit | Nicotine | 6671 | Alcohol |
| Vomiting | Promethazine | 7970 | Alcohol |
| Warts | Imiquimod | 4957 | Alcohol |
| Wound healing and | Aloe Vera gel | 312 | Alcohol |
| bed sores | | | |
| Atherosclerosis | CaNa₂EDTA | 3555 | Glycerol + Water |

In situations where a substance or a group of substances is only slightly soluble in the plasticizer formulation, the at least one substance may be added in a quantity such that a saturated solution is formed with undissolved at least one substance in contact with the saturated solution. This undissolved portion may then continue to dissolve as the substance in the solution is depleted by diffusion into the skin, thereby providing constant concentration of soluble at least one substance in the adhesive patch. Further, where the medication may be fully soluble in a plasticizer such as glycerol, a modified formulation containing, for example, glycerol and dioctyl phthalate can be used to provide the soluble/insoluble substance reservoir.

Zinc salicylate is both an antiseptic and a pain killer that is soluble in alcohol and glycerol. Zinc and other divalent metals such as manganese (See, e.g., Corbin et al. in “Metal Chelation and Inhibition of Bacterial Growth in Tissue Abscesses,” SCIENCE 319, pp. 962-5, 15 Feb. 2008.), silver powder and silver ions are useful as disinfectants and anti-fungal agents. Organic esters of these metals are generally soluble in alcohols and are effective components of topical treatments. Zinc bacitracin is a recognized anti-bacterial, zinc chloride and iodide are known antiseptics, and zinc proprionate is a topical anti-fungal. These compounds are sufficiently soluble in the plasticizers of the present invention to be used as topical medications.

### Example 3

Ammonium hydroxide is a good neutralizer for the acids of poison ivy and poison sumac. It is believed by the present inventor that alkaline additions to salicylates and other pain killers should provide effective patches or lotions. The zinc salicylate mentioned above may also be useful for this purpose, perhaps with slight additional alkalinity. Trolamine salicylate has pH of 10 at 1% concentration in water and is believed by the present inventor to be of comparable alkalinity and may be an effective neutralizer and painkiller for treatment of topical acids such as poison ivy, oak and sumac, either as a cream or in an adhesive composition. The reaction product of acetylsalicylic acid with triethanolamine is expected to perform the same function in treatments of such skin poisons.

### Example 4

A biodegradable, antibiotic, pressure-sensitive tape has been made using a combination of PVP, glycerin, Bacitracin and a nonwoven web of polylactic acid (Unitika #G0203WTO). This tape can be used within the body for quickly patching or wrapping wet organs, and it can be easily stitched in place if desired. All of the components of this tape are biodegradable.

### Example 5

Fosamax™ is generally administered as a pill or as an aqueous solution for treatment or prevention of osteoporosis at concentrations of about 70 mg/week. Fosamax™ has disadvantages of esophageal reflux ulceration and problems in the gastrointestinal tract. Being very polar, it has low permeability through the lipid walls of the GI tract into the blood. In U.S. Patent Application Publication No. 2006/0068010, “Method for Improving the Bioavailability of Orally Delivered Therapeutics,” it is reported that the bioavailability of orally administered (pill or solution) Fosamax™ is only 0.64% compared to the intravenous form. It is believed by the present inventor that a medicated patch using the pressure-sensitive adhesive composition of the present invention as a delivery vehicle can deliver up to 70 mg of sodium alendronate per week through the skin, thereby eliminating the scheduling of an early rising and a delayed breakfast, as is most common.

### Example 6

Chelation treatment is known to effectively remove lead ions from the human body, and, concurrently remove calcium from atherosclerotic plaque deposits in arteries. The National Institutes of Health is currently evaluating the effect chelation therapy on ischemic disease. Participants in these trials are receiving 40 infusions of calcium disodium ethylenedinitrilotetracetic acid solutions. The first 30 infusions are performed over a period of 30 weeks, taking 3 hours per infusion. Transdermal chelate treatment using a patch may be more efficient in both time and cost, and could be monitored as effectively as vascular infusion. A formulation has been prepared that may be effective for transdermally deliver this chelate. Although the solubility of calcium disodium EDTA is listed hereinabove as 10% in water and insoluble in alcohol, it was found to be 20% soluble in a mixture of water and glycerol having 1:3 ratio by weight. Since a relatively thick adhesive layer will be required to deliver the anticipated amount of medication to the skin and, as such, can be more readily removed in a shower than is desirable, a waterproof backing and a perimeter of a conventional waterproof pressure-sensitive adhesive around a patch of the water-soluble adhesive is desirable. This patch might be placed on the inner thigh over the femoral artery and femoral vein for most efficient delivery to blood and to the heart.

The following formulation was prepared to provide a solution that has a water/glycerol ratio in equilibrium with an atmosphere about 50% relative humidity:

| | | |
|---|---|---|
| Calcium disodium EDTA (Aldrich # 340073): | 5 | g; |
| Water: | 5 | g; |
| Glycerol: | 15 | g; |
| Polyvinylpyrrolidone, MW: 1.3 million Daltons: | 15 | g; |
| Silica aerogel M-5: | 1 | g; |
| Denatured alcohol: | 30 | g; and |
| Green food coloring solution (Kroger): | 1 | g. |

The chelate was soluble in the water/glycerol mixture and the complete mixture was an effective pressure-sensitive adhesive. The mixture was spread onto release paper where it was allowed to dry; it was then covered with a polyethylene film for transfer to the skin. A patch (area: 38.5 sq.cm. containing about 370 mg of the chelate) of this pressure-sensitive adhesive was worn by the inventor for more than 48 h. A green coloration of the skin implied transfer of the solution to the patient.

Effective patches may include greater than 1% chelating agent in a matrix of between 25% and 70% polyvinylpyrrolidone, between 10% and 50% glycerol, and between 2% and 15% of water on a carrier film having water vapor permeance of less than 500 g/m² atm.·day.

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A pressure-sensitive adhesive composition consisting essentially of: polyvinylpyrrolidone, at least one polar plasticizer and at least one substance to be delivered onto the skin or transdermally thereto.

2. The pressure-sensitive adhesive composition of claim 1, wherein the at least one plasticizer is chosen from polyols, aliphatic carbonates, and mixtures thereof.

3. The pressure-sensitive adhesive composition of claim 1, wherein the at least one substance is soluble in the mixture of polyvinylpyrrolidone and the at least one polar plasticizer.

4. The pressure-sensitive adhesive composition of claim 1, wherein the at least one substance forms a saturated solution with the mixture of polyvinylpyrrolidone and the at least one polar plasticizer.

5. The pressure sensitive adhesive composition of claim 1, wherein the at least one substance is chosen from analgesic, antiseptic, antiviral, chemotherapeutic, steroidal, fungicidal, hormonal, chelating, and oxidizing materials, and mixtures thereof.

6. The pressure-sensitive adhesive composition of claim 1, wherein the at least one substance is prepared by reacting equimolar quantities of triethanolamine with acetylsalicylic acid.

7. A pressure-sensitive adhesive composition comprising: polyvinylpyrrolidone as the principal pressure-sensitive adhesive, at least one polar plasticizer and at least one substance to be delivered onto the skin or transdermally thereto.

8. The pressure-sensitive adhesive composition of claim 7, wherein the at least one plasticizer is chosen from polyols, aliphatic carbonates, and mixtures thereof.

9. The pressure-sensitive adhesive composition of claim 7, wherein the at least one substance is soluble in the mixture of polyvinylpyrrolidone and the at least one polar plasticizer.

10. The pressure-sensitive adhesive composition of claim 7, wherein the at least one substance forms a saturated solution with the mixture of polyvinylpyrrolidone and the at least one polar plasticizer.

11. The pressure sensitive adhesive composition of claim 7, wherein the at least one substance is chosen from analgesic, antiseptic, antiviral, chemotherapeutic, steroidal, fungicidal, hormonal, chelating, and oxidizing materials, and mixtures thereof.

12. The pressure-sensitive adhesive composition of claim 7, wherein the at least one substance comprises the reaction product of triethanolamine with acetylsalicylic acid.

13. A method for delivering a substance onto the surface of skin or transdermally thereto, comprising the steps of:
forming a mixture of the at least one substance with a pressure-sensitive adhesive composition consisting essentially of: polyvinylpyrrolidone, and at least one polar plasticizer; and
applying the mixture to the surface of the skin.

14. The method of claim 13, wherein the at least one plasticizer is chosen from polyols, aliphatic carbonates, and mixtures thereof.

15. The method of claim 13, wherein the at least one substance is soluble in the mixture of polyvinylpyrrolidone and the at least one polar plasticizer.

16. The method of claim 13, wherein the at least one substance forms a saturated solution with the mixture of polyvinylpyrrolidone and the at least one polar plasticizer.

17. The method of claim 13, wherein the substance is chosen from analgesic, antiseptic, antiviral, chemotherapeutic, steroidal, fungicidal, hormonal, chelating, and oxidizing materials, and mixtures thereof.

18. The method of claim 13, further comprising the step of covering the mixture, forming thereby a transdermal patch.

19. The method of claim 18, wherein said step of covering the mixture is achieved using a microporous polymeric backing material.

20. The method of claim 19, wherein the polymeric backing material is biodegradable.

21. A method for delivering a substance onto the surface of skin or transdermally thereto, comprising the steps of:
forming a mixture of the at least one substance with a pressure-sensitive adhesive composition comprising: polyvinylpyrrolidone as the principal pressure-sensitive adhesive material, and at least one polar plasticizer; and
applying the mixture to the surface of the skin.

22. The method of claim 21, wherein the at least one plasticizer is chosen from polyols and aliphatic carbonates, and mixtures thereof.

23. The method of claim 21, wherein the at least one substance is soluble in the mixture of polyvinylpyrrolidone and the at least one polar plasticizer.

24. The method of claim 21, wherein the at least one substance forms a saturated solution with the mixture of polyvinylpyrrolidone and the at least one polar plasticizer.

25. The method of claim 21, wherein said substance is chosen from analgesic, antiseptic, antiviral, chemotherapeutic, steroidal, fungicidal, hormonal, and oxidizing materials, chelating, and mixtures thereof.

26. The method of claim 21, further comprising the step of covering the mixture, forming thereby a transdermal patch.

27. The method of claim 26, wherein said step of covering the mixture is achieved using a microporous polymeric backing material.

28. The method of claim 27, wherein the polymeric backing material is biodegradable.

29. A pressure-sensitive adhesive composition comprising: polyvinylpyrrolidone, less than 2% by weight of polyvinyl alcohol, at least one polar plasticizer, and at least one substance to be delivered onto skin or transdermally thereto.

30. The pressure-sensitive adhesive composition of claim 29, wherein the at least one plasticizer is chosen from polyols, aliphatic carbonates, and mixtures thereof.

31. The pressure-sensitive adhesive composition of claim 29, wherein the at least one substance is soluble in the mixture of polyvinylpyrrolidone, polyvinyl alcohol and the at least one polar plasticizer.

32. The pressure-sensitive adhesive composition of claim 29, wherein the at least one substance forms a saturated solution with the mixture of polyvinylpyrrolidone, polyvinyl alcohol and the at least one polar plasticizer.

33. The pressure-sensitive adhesive composition of claim 29, wherein the substance is chosen from analgesic, antiseptic, antiviral, chemotherapeutic, steroidal, fungicidal, hormonal, chelating, and oxidizing materials, and mixtures thereof.

34. The pressure-sensitive adhesive composition of claim 7, wherein the at least one substance comprises the reaction product of triethanolamine with acetylsalicylic acid.

35. A method for delivering a substance onto the surface of skin or transdermally thereto, comprising the steps of:
forming a mixture of the at least one substance with a pressure-sensitive adhesive composition comprising: polyvinylpyrrolidone, less than 2% by weight of polyvinyl alcohol, and at least one polar plasticizer; and
applying the mixture to the surface of the skin.

36. The method of claim 35, wherein the at least one plasticizer is chosen from polyols, aliphatic carbonates, and mixtures thereof.

37. The method of claim 35, wherein the at least one substance is soluble in the mixture of polyvinylpyrrolidone, polyvinyl alcohol and the at least one polar plasticizer.

38. The method of claim 35, wherein the at least one substance forms a saturated solution with the mixture of polyvinylpyrrolidone, polyvinyl alcohol and the at least one polar plasticizer.

39. The method of claim 35, wherein the substance is chosen from analgesic, antiseptic, antiviral, chemotherapeutic, steroidal, fungicidal, hormonal, chelating and oxidizing materials, and mixtures thereof.

40. The method of claim 35, further comprising the step of covering the mixture, forming thereby a transdermal patch.

41. The method of claim 40, wherein said step of covering the mixture is achieved using a microporous polymeric backing material.

42. The method of claim 41, wherein the polymeric backing material is biodegradable.

43. A bandage comprising a pressure-sensitive adhesive composition consisting essentially of: polyvinylpyrrolidone, at least one polar plasticizer and at least one substance to be delivered onto the skin or transdermally thereto.

44. A bandage comprising a pressure-sensitive adhesive composition comprising: polyvinylpyrrolidone as the principal pressure-sensitive adhesive, at least one polar plasticizer and at least one substance to be delivered onto the skin or transdermally thereto.

45. A pressure-sensitive adhesive composition comprising between 25 wt. % and 70 wt. % of polyvinylpyrrolidone; between 10 wt. % and 50 wt. % of at least one plasticizer having a solubility parameter exceeding 20 MPa^{0.5} and a normal boiling temperature exceeding 150° C.; between 2 wt. % and 15 wt. % of water; and greater than 1% of at least one chelating agent.

46. The pressure-sensitive adhesive composition of claim 45, further comprising a backing having water vapor permeance of less than 500 g/m² atm.·day.

47. The pressure-sensitive adhesive composition of claim 45, wherein the polyvinylpyrrolidone has a molecular weight greater than 25,000 Daltons, and the at least one plasticizer comprises glycerol.
